(19) United States
(12) Patent Application Publication
(10) Pub. No.: US 2020/0383946 A1
(43) Pub. Date: Dec. 10, 2020

(54) LIPOIC ACID FORMULATIONS

(71) Applicant: Aciont Inc., Salt Lake City, UT (US)

(72) Inventors: John W. Higuchi, Salt Lake City, UT (US); Kongnara Papangkorn, Sandy, UT (US)

(73) Assignee: Aciont Inc., Salt Lake City, UT (US)

(21) Appl. No.: 16/869,541

(22) Filed: May 7, 2020

Related U.S. Application Data
(60) Provisional application No. 62/844,457, filed on May 7, 2019.

(51) Int. Cl. A61K 31/385; A61K 9/00; A61K 9/08

(52) U.S. Cl. CPC A61K 31/385; A61K 9/08; A61K 9/0048

(57) ABSTRACT

A lipoic acid formulation can include water and an amount of a lipoic acid agent dissolved in the water. In some examples, a buffering agent can also be included in the formulation in an amount sufficient to dissolve the lipoic acid agent in the water. The lipoic acid formulation can generally have a pH of from about 6 to about 8.

### PRIORITY DATA

This application claims the benefit of U.S. Provisional Patent Application Ser. No. 62/844,457 filed on May 7, 2019, which is incorporated herein by reference.

### BACKGROUND

Alpha lipoic acid (ALA) is an organosulfur compound derived from caprylic acid (octanoic acid). It is produced in-vivo in animals under normal conditions and is essential for aerobic metabolism. It is also manufactured and is available as a dietary supplement in some countries where it is marketed as an antioxidant, and is available as a pharmaceutical drug in other countries. ALA is believed to help prevent certain kinds of cell damage in the body and to restore vitamin levels for vitamins such as vitamin C and vitamin E. There is also some evidence that alpha-lipoic acid can improve the function of neurons in diabetics. As such, alpha-lipoic acid is commonly administered orally to treat diabetes and associated nerve-related symptoms of diabetes. Alpha-lipoic acid is also used in the body to break down carbohydrates to make energy for the body.

### BRIEF DESCRIPTION OF THE DRAWINGS

For a fuller understanding of the nature and advantage of the present invention, reference is being made to the following detailed description of preferred embodiments and in connection with the accompanying drawings, in which:

FIG. 1a illustrates a front cross-sectional view of a non-invasive ocular drug delivery device, in accordance with some examples of the present disclosure.

FIG. 1b illustrates a bottom view of the non-invasive ocular drug delivery device of FIG. 1a.

FIG. 2a illustrates a front cross-sectional view of a non-invasive ocular drug delivery device, in accordance with other examples of the present disclosure.

FIG. 2b illustrates a bottom perspective view of the non-invasive ocular drug delivery device of FIG. 2a.

FIG. 2c illustrates a bottom view of the non-invasive ocular drug delivery device of FIG. 2c.

FIG. 3a illustrates a perspective view of a non-invasive ocular drug delivery device, in accordance with yet other examples of the present disclosure.

FIG. 3b illustrates a side cross-sectional view of the non-invasive ocular drug delivery device of FIG. 3a.

FIG. 3c illustrates a top view of the non-invasive ocular drug delivery device of FIG. 3a.

FIG. 3d illustrates a bottom view of the non-invasive ocular drug delivery device of FIG. 3a.

FIG. 4 illustrates a side cross-sectional view of the device of FIG. 3a attached to an eye, in accordance with some examples of the present disclosure.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a polymer” can include a plurality of such polymers.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art. For example, a composition that has “increased” ability to dissolve lipoic acid is able to solubilize a greater amount of lipoic acid as compared to a similar composition which does not achieve such results.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be lipoic acid. The terms “additional active agent,” “supplemental active agent,” “secondary active agent,” and the like can be used interchangeably and refer to a compound, molecule, or material other than lipoic acid that has physiologic activity when administered to a subject in an effective amount.

As used herein, the term “threshold dose” refers to an amount of a therapeutic agent which, when administered to a subject, is sufficient to achieve an intended therapeutic or physiological effect. Thus, a “threshold dose” refers to a non-toxic, but sufficient dose of a therapeutic agent, to achieve therapeutic results in treating a condition for which the therapeutic agent is known to be effective. It is understood that various biological factors may affect the ability of a therapeutic agent to perform its intended task. Therefore, a “threshold dose” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a threshold dose is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, a “dosing regimen” or “regimen” such as an “initial dosing regimen” or “starting dose” or a “maintenance dosing regimen” refers to how, when, how much, and for how long a dose of the compositions of the present invention can be administered to a subject. For example, an initial or starting dose regimen for a subject may provide for a total daily dose of 600 mg administered in two divided doses at least 12 hours apart (e.g. once with breakfast and once with dinner) with meals repeated daily for 30 days.

As used herein, “daily dose” refers to the amount of active agent (e.g. lipoic acid) administered to a subject over a 24 hour period of time. The daily dose can be administered two or more administrations during the 24 hour period. In one embodiment, the daily dose provides for two administrations in a 24 hour period. With this in mind, an “initial dose” or initial daily dose” refers to a dose administered during the initial regimen or period of a dosing regimen.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine.

As used herein, “lipoic acid,” “alpha lipoic acid” and the like may be used interchangeably and refer to a chemical compound known as α-lipoic acid, alpha lipoic acid, and thioctic acid and represented by the general structure:

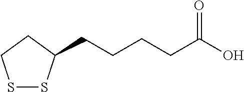 [SMILES: O=C(O)CCCC[C@@H]1CCSS1]

As used herein, “lipoic acid agent” refers to not only lipoic acid per se, but also includes related compounds, such as isomers, prodrugs, salts, metabolites, and derivatives thereof, etc. As such, instances of the term “lipoic acid agent” occurring in this written description are to be understood to provide express support for the above-recited lipoic acid compound per se, as well as related compounds as would be recognized by one of ordinary skill in the art.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 milligrams to about 80 milligrams” should also be understood to provide support for the range of “50 milligrams to 80 milligrams.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The present disclosure describes lipoic acid formulations, ophthalmic compositions, therapeutic systems, and methods of treatment, such as methods of reducing disulfide bonds in a lens of an eye. It is also noted that when discussing lipoic acid formulations, ophthalmic compositions, therapeutic systems, and methods of treatment described herein, these relative discussions can be considered applicable to the other examples, whether or not they are explicitly discussed in the context of that example. Thus, for example, in discussing a lipoic acid agent related to lipoic acid formulation, such disclosure is also relevant to and directly supported in the context of the ophthalmic compositions, therapeutic systems, and methods of treatment described herein, and vice versa.

In further detail, lipoic acid is generally understood to be minimally soluble in water (e.g. less than 1 mg/ml). As such, attempts have been made to stabilize lipoic acid with stabilizing agents or co-solvents and/or to esterify lipoic acid to increase the solubility thereof in water. The present disclosure describes lipoic acid formulations having a high concentration of a lipoic acid agent that are achievable by controlling the pH of the formulation without the need for a co-solvent, a stabilizing agent, a surfactant, or the like. Further, there is no need to modify the lipoic acid agent via esterification or the like to form a prodrug thereof. However, while these additional components and/or modifications are not necessary, they can also optionally be used within the scope of the present disclosure as well and are not intended to be excluded unless otherwise specified.

Thus, by controlling the pH of the lipoic acid formulation, a lipoic acid (LA) agent can be dissolved in water in an amount greater than or equal to about 35 mg/ml at a temperature of from about 20° C. to about 28° C. In other examples, the LA agent can be dissolved in water in an amount greater than or equal to about 40 mg/ml at a temperature of from about 20° C. to about 28° C. In still other examples, the LA agent can be dissolved in water in an amount greater than or equal to about 50 mg/ml at a temperature of from about 20° C. to about 28° C. In yet other examples, the LA agent can be dissolved in water in an amount greater than or equal to about 60 mg/ml at a temperature of from about 20° C. to about 28° C. In additional examples, the LA agent can be dissolved in water in an amount greater than or equal to about 65 mg/ml at a temperature of from about 20° C. to about 28° C. In still additional examples, the LA agent can be dissolved in water in an amount greater than or equal to about 70 mg/ml at a temperature of from about 20° C. to about 28° C. In yet additional examples, the LA agent can be dissolved in water in an amount greater than or equal to about 75 mg/ml at a temperature of from about 20° C. to about 28° C. In further examples, the LA agent can be dissolved in water in an amount greater than or equal to about 80 mg/ml at a temperature of from about 20° C. to about 28° C. In still further examples, the LA agent can be dissolved in water in an amount greater than or equal to about 90 mg/ml at a temperature of from about 20° C. to about 28° C. In yet further examples, the LA agent can be dissolved in water in an amount greater than or equal to about 100 mg/ml at a temperature of from about 20° C. to about 28° C. In still additional examples, the LA agent can be dissolved in water in an amount greater than or equal to about 110 mg/ml at a temperature of from about 20° C. to about 28° C.

The pH of the ophthalmic composition can generally be maintained from about 6 to about 8. However, in some examples, the pH of the ophthalmic composition can be maintained from about 6.5 to about 7.8. In other examples, the pH can be maintained from about 7 to about 8, from about 6.8 to about 7.8, or from about 7.2 to about 8.2. In some examples, generally the pH can be maintained using a suitable pH adjuster, such as hydrochloric acid, phosphoric acid, sodium hydroxide, the like, or a combination thereof.

While not expressly required, in some examples it can be valuable to employ a buffering agent in the lipoic acid formulation to help maintain the pH within a suitable range. A variety of suitable buffering agents can be used. Non-limiting examples can include a phosphate buffering agent, a borate buffering agent, a citrate buffering agent, a tromethamine buffering agent, a histidine buffering agent, the like, or a combination thereof. In some examples, the buffering agent can be present in the lipoic acid formulation in an amount from about 0.001 molar (M) to about 0.5 M. In other examples, the buffering agent can be present in the lipoic acid formulation in an amount from about 0.001 M to about 0.15 M. In still other examples, the buffering agent can be present in the lipoic acid formulation in an amount from about 0.005 M to about 0.05 M, from about 0.01 M to about 0.1 M, or from about 0.01 M to about 0.05 M.

It is further noted that a variety of LA agents can also be employed in the lipoic acid formulation. As various LA agents can have differing solubilities in water, the ideal pH of the lipoic acid formulation, appropriate buffering agents, use of optional additional agents, and the like can also vary to some degree depending on the particular LA agent(s) employed. Non-limiting examples of suitable LA agents can include alpha-lipoic acid, dihydrolipoic acid, dihydrolipoate, 5-(1,2-thiaselenolan-5-yl) pentanoic acid, 5-(1,2-thiaselenolan-3-yl) pentanoic acid, choline esters and/or derivatives of lipoic acid, such as alpha lipoic acid, 6,8-dimercaptooctanoic acid, dihydrolipoate, 5-(1,2-thiaselenolan-5-yl)pentanoic acid or 5-(1,2-thiaselenolan-3-yl)pentanoic acid, a salt thereof, an enantiomer thereof, a prodrug thereof, the like, or a combination thereof.

The lipoic acid formulations described herein can be used for research, as therapeutic compositions (e.g. enteral, parenteral, topical) for treatment of a variety of conditions (e.g. diabetes, neurological disorders, etc.), as nutritional supplements, or a variety of other suitable uses. In some examples, where the lipoic acid formulation is a therapeutic composition, a nutritional supplement, or the like, the lipoic acid formulation can include an additional active agent. Non-limiting examples of additional active agents can include a miotic agent, a cycloplegic agent, a non-steroidal anti-inflammatory agent, an antioxidant, a vitamin, the like, or a combination thereof. Non-limiting examples of miotic agents can include carbamoylcholine, pilocarpine, physostigmine, echothiophate, methacholine, moxisylyte, acetylcholine, the like, or a combination thereof. Non-limiting examples of cycloplegic agents can include tropicamide, cyclopentolate, ephedrine, homatropine, phenylephrine, atropine, scopolamine, methscopolamine, the like, or a combination thereof. Non-limiting examples of non-steroidal anti-inflammatory agents can include ketorolac tromethamine, flurbiprofen sodium, diclofenac sodium, bromfenac, nepafenac, amfenac, aspirin, ibuprofen, naproxen, nabumetone, the like, or a combination thereof. Non-limiting examples of antioxidants can include vitamin C, vitamin E, lutein, zeaxanthin, glutathione, edaravone, N-acetylcysteine, diosmin, hesperidin, oxerutins, baicalein, catechins, the like, or a combination thereof. Non-limiting examples of vitamins can include vitamin A, thiamine, riboflavin, niacin, pantothenic acid, biotin, folate, vitamin B6, vitamin B12, vitamin C, vitamin D, vitamin E, vitamin K, the like, or a combination thereof. Various other additional active agents can also be used, such as steroids (e.g. dexamethasone, hydrocortisone, loteprednol, prednisolone, the like, or a combination thereof), antibiotics (e.g. besifloxacin, ciprofloxacin, moxifloxacin, polymyxin B, bacitracin, neomycin, tobramycin, erythromycin, the like, or a combination thereof), anti-glaucoma agents (e.g. levobunolol hydrochloride, timolol maleate, latanoprost, bimatoprost, brimonidine, dorzolamide, the like, or a combination thereof), etc. In some examples, the lipoic acid formulation does not include an additional active agent.

In some specific examples, the lipoic acid formulation can be formulated as an ophthalmic composition, such as for use as a medicament for the treatment of an ocular condition. For example, disulfides presumed to be developed through aging and environmental factors may increase protein crosslinking in the lens of the eye, which can stiffen the lens. The lens stiffness is believed to be responsible for the ocular condition presbyopia. Without being bound by a particular theory, it is believed that lipoic acid, delivered to the lens of the eye if sufficient amounts, can decrease the amount of disulfide crosslinking in the lens, reduce the stiffness of the lens, and increase the elasticity of the lens. Thus, lipoic acid can be an effective active agent in the treatment of presbyopia, for example.

Thus, in some examples, the ophthalmic composition can include an amount of LA agent sufficient to reduce disulfide bonds in a lens of an eye when administered topically to the eye. The LA agent can be included in a pharmaceutically acceptable aqueous carrier. Generally, the ophthalmic composition can have a pH of from about 6 to about 8 and a viscosity of less than or equal to 50 centipoise (cps). In some examples, the ophthalmic composition can include an amount of buffering agent sufficient to solubilize or to help solubilize the amount of LA agent in the pharmaceutically acceptable aqueous carrier.

In further detail, the LA agent can typically be present in the ophthalmic composition in an amount from about 5 mg/ml to about 150 mg/ml. In some examples, the LA agent can be present in the ophthalmic composition in an amount from about 5 mg/ml to about 25 mg/ml, from about 20 mg/ml to about 50 mg/ml, from about 40 mg/ml to about 70 mg/ml, from about 60 mg/ml to about 90 mg/ml, from about 80 mg/ml to about 110 mg/ml, or from about 100 mg/ml to about 130 mg/ml. In some specific examples, the LA agent can be present in the ophthalmic composition in an amount from about 5 mg/ml to about 15 mg/ml, from about 10 mg/ml to about 20 mg/ml, from about 15 mg/ml to about 25 mg/ml, or from about 20 mg/ml to about 30 mg/ml.

As described previously, the viscosity of the ophthalmic composition can typically be less than about 50 cps. In other examples, the viscosity of the ophthalmic composition can be less than about 40 cps, 30 cps, or 20 cps. In some specific examples, the viscosity of the ophthalmic composition can be from about 5 cps to about 50 cps, from about 10 cps to about 35 cps, or from about 15 cps to about 25 cps. Having a reasonably low viscosity can help facilitate delivery of the ophthalmic composition via a variety of delivery modalities and ocular delivery devices. Typically, the ophthalmic composition is substantially free of gelling agents or thickening agents included in many eye drops, as described in greater detail below.

In some examples, the ophthalmic composition can include a tonicity agent. Non-limiting examples can include sodium chloride, potassium chloride, calcium chloride, magnesium chloride, mannitol, sorbitol, dextrose, glycerin, propylene glycol, ethanol, trehalose, the like, or combinations thereof. The tonicity of the ophthalmic composition can typically be from about 250 mOsm/kg to about 500 mOsm/kg. In some specific examples, the tonicity can be from about 250 mOsm/kg to about 400 mOsm/kg. In yet other examples, the tonicity of the composition can be from about 250 mOsm/kg to about 325 mOsm/kg, from about 300 mOsm/kg to about 375 mOsm/kg, or from about 350 mOsm/kg to about 425 mOsm/kg. In still other examples, the tonicity can be from about 400 mOsm/kg to about 500 mOsm/kg.

In some examples, the ophthalmic composition can include a preservative. Where this is the case, a variety of preservatives can be included. Non-limiting examples can include a benzalkonium halide, polyquaternium-1, chlorine dioxide, benzehonium chloride, chlorobutanol, phenylmercuric acetate, phenylmercuric nitrate, thimerosal, parahydroxybenzoates, cetrimonium, phenylethylalcohol, polyhexamethylene biguanide, sodium perborate, stabilized oxychloro complex, the like, or a combination thereof.

In some specific examples, the composition can include a chelating agent. Non-limiting examples can include edetate disodium dihydrate, edetic acid, ethylene diamine, porphine, the like, or combinations thereof. Where a chelating agent is included in the ophthalmic composition, it can typically be present in an amount of from about 0.001% w/v to about 0.1% w/v, or from about 0.005% w/v to about 0.05% w/v. However, in some examples, the composition does not include a chelating agent. For example, in some cases, the lipoic acid agent can be sourced or prepared to have a very low concentration of metal ions. Alternatively, or additionally, in some examples, the container can impart a very low amount of metal ions to the composition. For example, in some cases, the container can be a plastic container, a glass container with an interior plastic coating, or the like, that does not impart significant amounts of metal ions. Thus, a chelating agent is not always desirable or necessary.

In some examples, the ophthalmic composition can include a stabilizing agent. Non-limiting examples of stabilizing agents can include a cyclodextrin, a surfactant, the like, or a combination thereof. However, a stabilizing agent is not always desirable or necessary. In some specific examples, the ophthalmic composition does not include stabilizing agent.

The ophthalmic composition can typically be particulate-matter-free or substantially particulate-matter-free. As used herein, the term “particulate-matter-free” or its grammatical equivalents such as “particle free” refer to the state in which the ophthalmic composition meets the USP requirements for particulate matter in ophthalmic compositions. See for example, USP, Chapter 789. One of skill in the art understands and knows how to assess whether a given composition meets USP particulate matter requirements. With this in mind, in some examples, the ophthalmic composition can include less than or equal to 50 particles per milliliter (mL) having a particle diameter greater than or equal to 10 μm. In still additional examples, the ophthalmic composition can include less than or equal to 5 particles per mL having a particle diameter greater than or equal to 25 μm. In yet additional examples, the ophthalmic composition can include less than or equal to 2 particles per mL having a particle diameter greater than or equal to 50 μm. These values can be determined using the Light Obscuration Particle Count Test, the Microscopic Particle Count Test, or both, as described in USP, Chapter 789.

Thus, in some examples, the ophthalmic composition can be an ophthalmic solution. As such, in some examples, the ophthalmic composition is not a gel, an ointment, a suspension, an emulsion, or the like. Further, in some examples, the ophthalmic composition can include limited amounts of excipients. As used herein, an “excipient” refers to components in the ophthalmic composition other than the lipoic acid agent and water. In some specific examples, the total amount of excipients in the ophthalmic composition can be less than 10% w/v. In some additional examples, the total amount of excipients in the ophthalmic composition can be less than 5% w/v, less than 3% w/v, or less than 1% w/v.

In some examples, the ophthalmic composition can be free or substantially free of preservative, such as those listed above. In some examples, the ophthalmic composition can be free or substantially free of additional antioxidant, other than the lipoic acid agent(s). Non-limiting examples of additional antioxidants can include sodium metabisulphite, sodium formaldehyde sulphoxylate, sodium sulphite, N-acetylcarnosine, L-carnosine, L-glutathione, cysteine, ascorbate, L-cysteine, and the like. In some additional examples, the ophthalmic composition can be free or substantially free of a buffering agent, such as those listed above. In still additional examples, the composition can be free or substantially free of polymer (e.g. a thickening agent, a gelling agent, or the like), such as cellulosic compounds (e.g. carboxymethyl cellulose, hydroxypropyl cellulose, hydroxyethyl cellulose, etc.), carbomers, polyvinyl alcohol, gelatin, polyvinyl pyrrolidone, polysaccharide thickeners (e.g. starches, vegetable gums, pectin, etc.), or the like. In some examples, the ophthalmic composition does not include surface-active agent or surfactant. Non-limiting examples of surface-active agents can include non-ionic surfactants (e.g. sorbitan oleates, polysorbates, polyoxyethylene ethers, etc.) anionic surfactants, and cationic surfactants. In some examples, the ophthalmic composition does not include a cyclodextrin, or the like. In some examples, the ophthalmic composition does not include a hydrocarbon or a sterol (e.g. cholesterol). In some examples, the ophthalmic composition does not include one or more of a preservative, an antioxidant, a buffering agent, a polymer, a surface active agent, a cyclodextrin, a hydrocarbon, or a sterol. In some examples, the ophthalmic composition does not include two or more of a preservative, an antioxidant, a buffering agent, a polymer, a surface active agent, a cyclodextrin, a hydrocarbon, or a sterol. In some examples, the ophthalmic composition does not include three or more of a preservative, an antioxidant, a buffering agent, a polymer, a surface active agent, a cyclodextrin, a hydrocarbon, or a sterol. In some examples, the composition does not include one or more of a polymer, a dendrimer, a micelle, a liposome, a nanoparticle, a surface-active agent, or the like.

In some examples, the ophthalmic composition can be sterile. A number of sterilization procedures can be used to sterilize the ophthalmic composition. Non-limiting examples of sterilization procedures can include EtO sterilization, gamma sterilization, E-beam sterilization, x-ray sterilization, vaporized hydrogen peroxide (VHP) sterilization, steam sterilization, dry-heat sterilization, filtration, the like, or combinations thereof.

In some examples, the ophthalmic composition can be supplied in an enclosed container, which can be a sterile container. Where this is the case, the container can include any suitable container. In some examples, the container can be made of a material such as glass, polyethylene, polypropylene, polyvinyl chloride, polycarbonate, the like, or a combination thereof. In some examples, the container can have a volume of from about 0.5 ml to about 10 ml, from about 0.5 ml to about 5 ml, or from about 0.75 ml to about 1.5 ml.

In some examples, the ophthalmic composition can be supplied with or in an ocular drug delivery device that is adapted to couple to an eye of a subject. In some examples, the device can include at least 50 μL, at least 100 μL, or at least 150 μL of the ophthalmic composition pre-loaded therein. In some examples, the device can include from about 50 L to about 5000 μL of the ophthalmic composition pre-loaded therein. In other examples, the device can include from about 100 μL to about 1000 μL of the ophthalmic composition pre-loaded therein. In yet other examples, the device can include from about 150 μL to about 500 μL of the ophthalmic composition pre-loaded therein. In some specific examples, the device can include from about 120 μL to about 300 μL of the ophthalmic composition pre-loaded therein.

A variety of suitable devices can be used. In some specific examples, the device can be a non-invasive ocular drug delivery device including a housing and an active agent matrix coupled to the housing.

In further detail, the housing of the non-invasive ocular drug delivery device is not particularly limited, other than it is adapted to couple to an eye of a subject. Thus, in some examples, the housing can couple directly to the eye, such as via negative pressure, surface tension, adhesives, the like, or combinations thereof. In yet other examples, the housing can be shaped to interface with the eye and can be held against the eye using positive pressure from eye lids, and/or straps, cords, scaffolding, adhesives, the like, or combinations thereof that are attached to a surface outside of the eye, but nonetheless hold the housing in place against the eye.

In some examples, the housing can be formed from a plurality of interconnecting pieces to prepare an integral housing. In yet other examples, the housing can be formed as a monolithic unit. Thus, in some cases, the housing can be formed from a mold or other suitable manufacturing process as a single monolithic unit without any need for further assembly or integration of additional components. In some specific examples, the monolithic unit can be formed of a molded elastomeric material, such as ethylene propylene diene monomer (EPDM), fluoroelastomers (e.g. FKMs, FFKMs, FEPMs, etc.), acrylonitrile-butadiene rubbers, silicones, the like, or combinations thereof. Whether the housing is formed of a molded material or not, the housing can include a variety of suitable materials, such as one or more of the elastomeric materials listed above, polyamides, polyesters, polyethylenes, polypropylenes, polycarbonates, polyurethanes, polytetrafluoroethylenes, metals, the like, or combinations thereof. In some specific examples, the housing can include or be formed of an EPDM material. In yet other examples, the housing can include or be formed of a fluoroelastomer material. In still other examples, the housing can include or be formed of an acrylonitrile-butadiene rubber. In yet additional examples, the housing can include or be formed of a silicone material.

In still additional examples, the housing can include or be formed of a translucent or transparent material. For example, many of the materials listed above can be prepared in a way so that they are translucent or transparent. Other translucent or transparent materials can also be used. In some examples, portions of the housing can be translucent or transparent while others are not. In yet other examples, portions of the housing can be translucent while other parts of the housing can be transparent. In some specific examples, at least a portion of the housing that covers the cornea can be translucent or transparent.

The geometry of the housing is not particularly limited, so long as the housing adequately interfaces with a surface of the eye to facilitate administration of an active agent. However, in some examples, the housing (or at least the portion of the housing that interfaces with the eye) can have an elliptical geometry. While the overall shape of the eye approaches a spherical geometry, the part of the eye that is visible generally has an elliptical shape. Thus, the housing (or at least the portion of the housing that interfaces with the eye) can be prepared so as to have an elliptical, or approximately elliptical, shape. In some examples, an elliptical shape can facilitate application of the device to the eye and maximize the comfort of the subject, while maintaining adequate surface coverage or interface area of the device with the eye to provide an adequate dose of an active agent in a timely manner. Where the device has an elliptical geometry, the device can typically have an aspect ratio (width to height) of from about 1.05:1 to about 1.4:1. In yet other examples, the device can have an aspect ratio of from about 1.10:1 to about 1.3:1. In still other examples, the device can have an aspect ratio of from about 1.15:1 to about 1.25:1.

In some specific examples, the housing can include a corneal dome shaped to cover the cornea of the eye. The corneal dome can generally be shaped to maintain a gap between a portion of the cornea and an inner surface of the corneal dome. This gap can also facilitate the comfort of the user while using the device. As is known to one skilled in the art, the cornea can be a very sensitive portion of the eye. As such, in some cases, it can facilitate user comfort by minimizing contact of the device with the cornea. In some examples, the gap between the portion of the cornea and the inner surface of the corneal dome can be at least 50 μm or at least 100 μm. In yet other examples, the gap between the portion of the cornea and the inner surface of the corneal dome can be at least 200 μm or at least 500 μm. In still other examples, the gap between the portion of the cornea and the inner surface of the corneal dome can be at least 1000 μm. The portion of the cornea where the gap is maintained can generally be at least 50% of the corneal surface area. Thus, for example, in some cases, a gap of at least 100 μm between an inner surface of the corneal dome and the cornea of the eye can be maintained over at least 50% of the corneal surface. In some examples, the portion of the cornea where the gap is maintained can be at least 60%, 70%, 80%, or 90% of the corneal surface area. In yet other examples, the gap can be maintained across the entire corneal surface area.

In some additional examples, the housing can include a corneal seal that is positioned to circumscribe the cornea and form a fluidic seal against the eye to minimize fluid transport across the corneal seal to the cornea when in use. It is noted that where the device does not include a corneal dome, the cornea can be exposed to ambient conditions. However, the corneal seal can still minimize fluid transport (e.g. from the active agent matrix, for example) across the surface of the eye to the cornea. Where the housing includes a corneal dome, the corneal seal can be disposed about a periphery of the corneal dome to minimize fluid transport to the cornea when in use. It is noted that when the diameter of the corneal seal becomes too large, it can be challenging to comfortably maintain the housing within the framework of the eyelids. Thus, the corneal seal can be shaped to maintain a seal about the cornea without excessively increasing the overall size of the housing. In some examples, the corneal seal can be shaped to maintain a distance from a perimeter of the cornea (i.e. the corneal seal is positioned exterior to the cornea so as to not contact the cornea) of from about 50 μm to about 5000 μm. In yet other examples, the corneal seal can be shaped to maintain a distance from a perimeter of the cornea of from about 500 μm to about 3000 μm. In still other examples, the corneal seal can be shaped to maintain a distance from a perimeter of the cornea of from about 1000 μm to about 2000 μm. In some specific examples, the corneal seal can be shaped to maintain a distance from a perimeter of the cornea of from about 50 μm to about 1000 μm, about 100 m to about 1500 μm, or about 300 μm to about 1200 μm.

In some further examples, the housing can include a scleral flange extending radially outward from the corneal seal. In some examples, where the housing (or at least the portion of the housing that interfaces with the eye) has an elliptical geometry, the scleral flange can have a shape that provides the elliptical geometry. In some examples, the scleral flange can be the portion of the housing to which the active agent matrix is attached. Where this is the case, the scleral flange can be shaped to maintain contact between the active agent matrix and the sclera of the eye when in use. The scleral flange can generally be shaped and positioned on the housing so as to cover a portion of the sclera of the eye without covering the cornea. Additionally, in some examples, the scleral flange, or other similar segment of the housing, can include a scleral lip or scleral seal about a perimeter of the portion of the device that interfaces with the eye. In some examples, the scleral lip or scleral seal can be shaped to facilitate retention of the active agent matrix to the housing, such as via friction fitting, nesting, clamping, or the like. In some examples, the scleral lip or scleral seal can additionally form a fluidic seal against the eye to minimize fluid transport across the scleral seal. In some examples, this can help concentrate delivery of the active agent to a specific region of the sclera and improve delivery of the active agent to the posterior segment of the eye.

In some examples, a pressure regulator can be operatively connected to the housing and adapted to induce negative pressure between the housing and the eye to couple the housing to the eye when in use. In some examples, the pressure regulator can form part of the housing, such as an integrated component of the housing or as part of a monolithic housing. In some examples, the pressure regulator can be a bulb, a pump, the like, or other suitable pressure regulator that can be operatively connected to the housing. The pressure regulator can generally be adapted to induce a negative pressure between the housing and the eye to couple the housing to the eye when in use. The negative pressure induced between the housing and the eye can be any pressure suitable to maintain the housing on the eye without significantly damaging the eye. In some examples, the pressure regulator can be adapted to induce a negative pressure of from about 0.98 atmospheres (atm) to about 0.1 atm between the housing and the eye. In yet other examples, the pressure regulator can be adapted to induce a negative pressure of from about 0.90 atm to about 0.3 atm. In still other examples, the pressure regulator can be adapted to induce a negative pressure of from about 0.8 atm to about 0.5 atm. In some examples, the pressure regulator can be adapted to reduce a pressure between the housing and the eye by an amount from about 0.1 atm to about 3 atm relative to atmospheric pressure. In yet other examples, the pressure regulator can be adapted to reduce a pressure between the housing and the eye by an amount from about 0.5 atm to about 1 atm relative to atmospheric pressure.

The active agent matrix can be coupled to the housing using any suitable coupling feature, such as an adhesive, stitching, friction-fitting, clips, clamps, magnets, snaps, hook and loop fasteners, the like, or combinations thereof. In some specific examples, the active agent matrix can be coupled to the housing via an adhesive. A variety of suitable adhesives can be used. Non-limiting examples can include a silicone adhesive, an acrylic adhesive, a polyurethane adhesive, the like, or combinations thereof. Further, the active agent matrix can generally be positioned to interface with the sclera of the eye, but not the cornea of the eye. In some examples, the active agent matrix can be formed of a plurality of segments that are positioned adjacent to one another to form an integral active agent matrix. In some specific examples, the active agent matrix can be formed from 2, 3, 4, or more individual segments positioned adjacent to one another. In some examples, the individual segments can be spaced apart from one another. In yet other examples, the individual segments can be positioned so that there is substantially no space between adjacent segments.

The active agent matrix can have a variety of suitable densities. In some specific examples, the active agent matrix can have a density of from about 0.15 grams/cubic centimeter (cc) to about 0.4 grams/cc prior to loading with the active agent composition. In yet other examples, the active agent matrix can have a density of from about 0.18 g/cc to about 0.35 g/cc prior to loading the active agent composition. In still other examples, the active agent matrix can have a density of from about 0.2 g/cc to about 0.31 g/cc prior to loading the active agent composition.

The active agent matrix can also have a variety of thicknesses. In some specific examples, the active agent matrix can have a thickness of from about 250 μm to about 600 m prior to loading with the active agent composition. In yet other examples, the active agent matrix can have a thickness of from about 300 μm to about 500 μm prior to loading with the active agent composition. In still other examples, the active agent matrix can have a thickness of from about 350 μm to about 450 μm prior to loading with the active agent composition. The post-loading thickness of the active agent matrix can typically be greater than the pre-loading thickness of the active agent matrix. For example, in some cases, the post-loading thickness can be from about 2 times to about 6 times the pre-loading thickness. In yet other examples, the post-loading thickness can be from about 3 times to about 5 times the pre-loading thickness.

The active agent matrix can have a variety of ocular surface areas or ocular interface areas (i.e. the area of the active agent matrix that interfaces with the eye). In some examples, the ocular surface area of the active agent matrix can be from about 50 mm² to about 300 mm². In some additional examples, the ocular surface area of the active agent matrix can be from about 75 mm² to about 250 mm². In yet other examples, the ocular surface area of the active agent matrix can be from about 100 mm² to about 200 mm².

In some examples, the non-invasive ocular delivery device can be a passive delivery device. As such, in some examples, the non-invasive ocular delivery device does not include an electrode or other electrical components used in an active delivery device. In some specific examples, the non-invasive ocular delivery device does not include an electrode or other electrical components adapted specifically for iontophoretic administration of the active agent. In other examples, the non-invasive ocular drug delivery device can include an electrode and/or other electrical components to adapt or configure the device for active administration of the active agent, such as iontophoretic delivery, electroporation, sonoporation, the like, or combinations thereof.

The present disclosure also describes various methods of treatment, such as methods of treating glaucoma, presbyopia, a cataract, anterior uveitis, dry eye, ocular hypertension, the like, or a combination thereof. In one specific example, the present disclosure describes methods of reducing disulfide bonds in a lens of an eye, which can also be employed in the treatment of a medical condition including glaucoma, presbyopia, a cataract, anterior uveitis, dry eye, ocular hypertension, the like, or a combination thereof. The methods of reducing disulfide bonds in a lens of an eye can include topically administering an LA agent to the eye of a subject in an extended or continuous dosing event. The extended or continuous dosing event does not include administration of an eye drop. Rather, the extended or continuous dosing event includes continuous delivery of an active agent over a period of time, as will be described in greater detail hereafter.

In some further examples, topical administration can include administering the LA agent to the eye via the sclera without topical administration via the cornea. In other words, in some examples, topical administration can include topical administration to the sclera without topical administration to the cornea. In some examples, topical administration can include administration to the cornea. In some further examples, topical administration can include coupling a therapeutic delivery device to the eye, such as a contact lens, therapeutic delivery device as described herein, or the like. Regardless of the site of administration, the LA agent can be ultimately delivered to a variety of ocular locations, such as one or more of the sclera, ciliary body, iris, aqueous humor, lens, anterior chamber, and cornea.

In some specific examples, the administration of the LA agent can be performed via passive administration. As such, LA agent can be topically administered to the eye and allowed to passively diffuse into the eye. In some examples, passive administration can employ penetration enhancers or other suitable delivery aids to increase the rate at which LA agent is delivered to the eye. In other examples, passive administration does not employ penetration enhancers or the like. In some specific examples, passive administration can be non-invasive passive administration.

In some examples, the administration of LA agent can be performed via active administration. Active administration can include iontophoresis, electroporation, ultrasound, microneedles, the like, or a combination thereof to actively deliver LA agent to the eye. However, it is noted that where active administration is non-invasive, microneedles or the other administration methods that are configured to pierce or puncture an ocular surface are not considered non-invasive administration techniques. As drug delivery methods employing iontophoresis, electroporation, ultrasound, or microneedles are generally known in the art, such methods will not be discussed in detail. However, it is to be understood that such methods, and other similar methods, are considered to be within the scope of the present disclosure. In some specific examples, active administration can include iontophoretic administration of LA agent to the eye. In yet other examples, active administration can include electroporation or electroporation-facilitated delivery of LA agent to the eye. In some examples, active administration can include ultrasound or ultrasound-facilitated delivery of LA agent to the eye. In some examples, active administration can employ microneedles to facilitate delivery of LA agent to the eye.

Whatever the mode of administration, LA agent can typically be administered via a therapeutically effective dosing regimen that includes one or more extended or continuous administration periods. More specifically, each administration event is typically performed for a continuous or consecutive period. Generally, the continuous period is less than one week. In some additional examples, the continuous period is less than or equal to 5 days, less than or equal to 3 days, or less than or equal to 1 day (i.e. 24 hours). In some specific examples, the consecutive period can be a period of from about 1 minute to about 30 minutes. In yet other examples, the consecutive period can be a period of from about 2 minutes to about 20 minutes, from about 3 minutes to about 15 minutes, from about 4 minutes to about 10 minutes, or from about 5 minutes to about 8 minutes. It is noted that the continuous or consecutive period can be adjusted based on the concentration of the LA agent. For example, where a longer administration event or administration period is desired, a lower concentration of LA agent can be used. Conversely, where a shorter administration event or administration period is desired, a greater concentration of LA agent can be used.

Thus, each administration event can be a sufficient continuous period of time to introduce a threshold dose of LA agent to the eye. In some examples, the threshold dose can be considerably higher than an amount administered via an eye drop. In some cases, the threshold dose can deliver at least about 5 times more LA agent to the eye than an eye drop. In some cases, the threshold dose can depend on the type and severity of the condition being treated, the specific individual being treated, etc. In some examples, the threshold dose can be an amount from about 0.005 mg to about 5 mg of LA agent. In yet other examples, the threshold dose can be an amount from about 0.01 mg to about 2.5 mg of LA agent. In still other examples, the threshold dose can be an amount from about 0.05 mg to about 1 mg of LA agent. In some specific examples, the threshold dose can be an amount from about 0.1 mg to about 0.5 mg of LA agent. In other specific examples, the threshold dose can be an amount from about 0.2 mg to about 1 mg, about 0.3 mg to about 2 mg, or about 0.25 mg to about 1.5 mg of LA agent. In yet other specific examples, the threshold dose can be an amount from about 0.01 mg to about 0.05 mg, about 0.05 mg to about 0.1 mg, about 0.1 mg to about 0.5 mg, about 0.5 mg to about 2.5 mg, or about 1 mg to about 5 mg.

Generally, topical administration via the continuous administration event is performed at a frequency of from once per day to once every 6 months during a treatment period. In other examples, topical administration via the continuous administration event is performed at a frequency of from once every two days to once every 10 days, from once every 7 days to once every 14 days, from once every 14 days to once every 28 days, from once per month to once every 3 months, or from once every three months to once every 6 months. In some specific examples, topical administration is performed no more than once per day.

Whatever the frequency of administration, the treatment period typically includes from 1 to 8 administration events. In some examples, the treatment period can include from 1 to 4 administration events, from 2 to 6 administration events, or from 4 to 8 administration events.

In some examples, the methods can further include administering an additional active agent, such as one or more of the additional active agents listed elsewhere herein. The additional active agent can be administered in a variety of ways. In some specific examples, the additional active agent can be administered topically. In some further examples, the additional active agent can be co-administered with the LA agent. For example, the additional active agent can be included in the lipoic acid formulation, can be co-loaded to a common active agent delivery device, or the like. In other examples, the additional active agent can be administered sequentially with the LA agent, which can be prior to administration of the LA agent, after administration of the LA agent, or both. Where this is the case, additional active agent can be administered in the same manner as the LA agent or by another suitable mode of administration. For example, in some cases, the additional active agent can be administered to the eye via an eye drop, intravitreal injection, implant, etc.

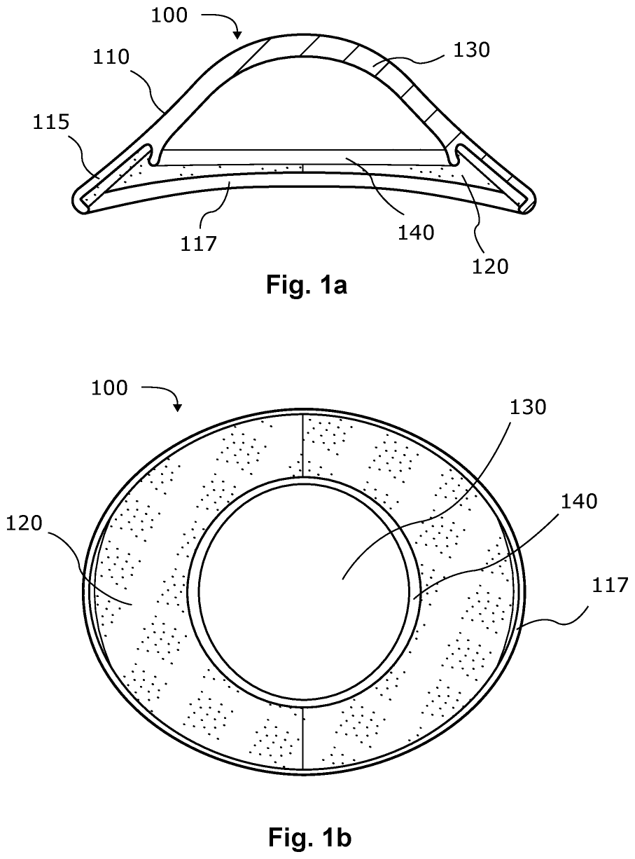

Turning now to the figures, FIGS. 1a and 1b illustrate one example of a non-invasive ocular drug delivery device 100 having a housing 110 and an active agent matrix 120 coupled thereto. In this particular example, the active agent matrix 120 includes two semicircle segments, but can include a single segment or other suitable number of segments. The housing 100 includes a corneal dome 130 shaped to cover a cornea of an eye. Additionally, the housing includes a corneal seal 140 positioned about a perimeter of the corneal dome 130 to form a fluidic seal against the eye when in use to minimize fluid transport into the corneal dome 130. The housing also includes a scleral flange 115 positioned to cover a portion of the sclera of an eye without covering the cornea. A scleral lip or scleral seal 117 is disposed about a perimeter of the scleral flange 115.

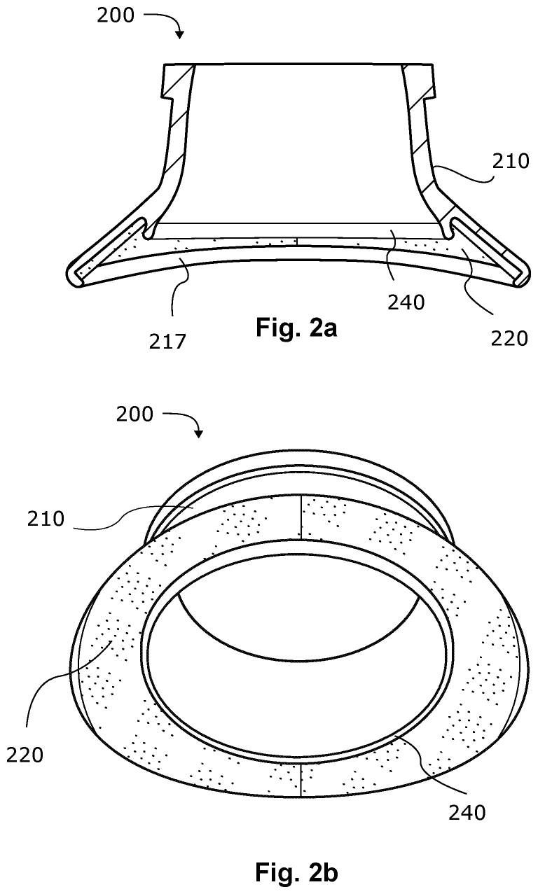

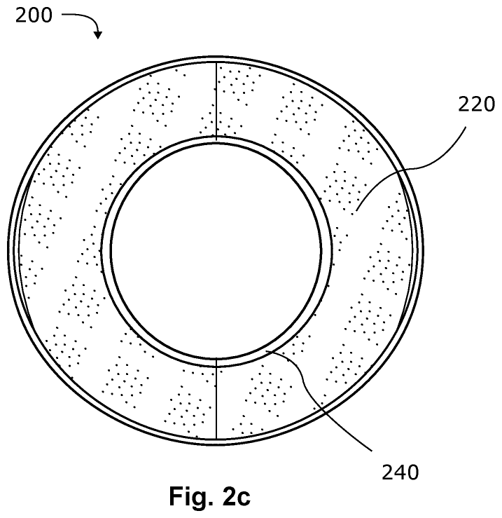

FIGS. 2a, 2b, and 2c illustrate an alternative example of a non-invasive ocular drug delivery device 200 having a housing 210 and an active agent matrix 220 coupled thereto. In this particular example, the housing 200 does not include a corneal dome. As such, the cornea of the eye can be exposed to ambient conditions during use of this particular example of the device 200. Nonetheless, the device 200 still includes a corneal seal 240 to minimize fluid transport across the surface of the eye to the cornea. This can minimize surface contact of the active agent with the sensitive cornea. The device 200 can also include a scleral lip or scleral seal 217 adapted to contain topical delivery of the active agent between the corneal seal 240 and the scleral seal 217.

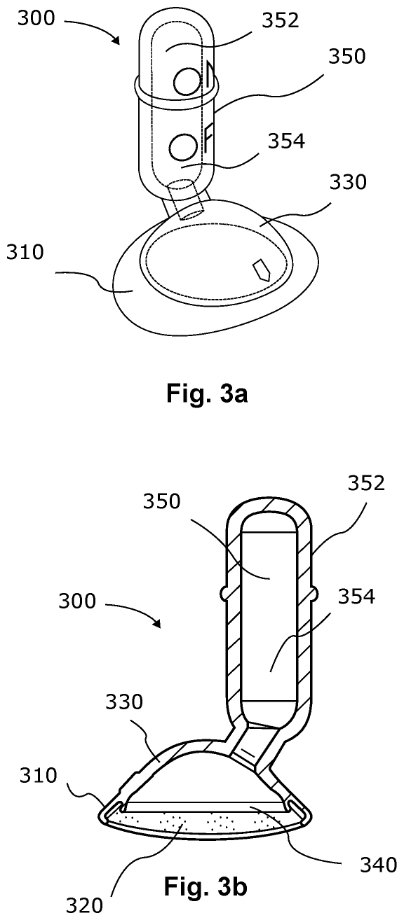

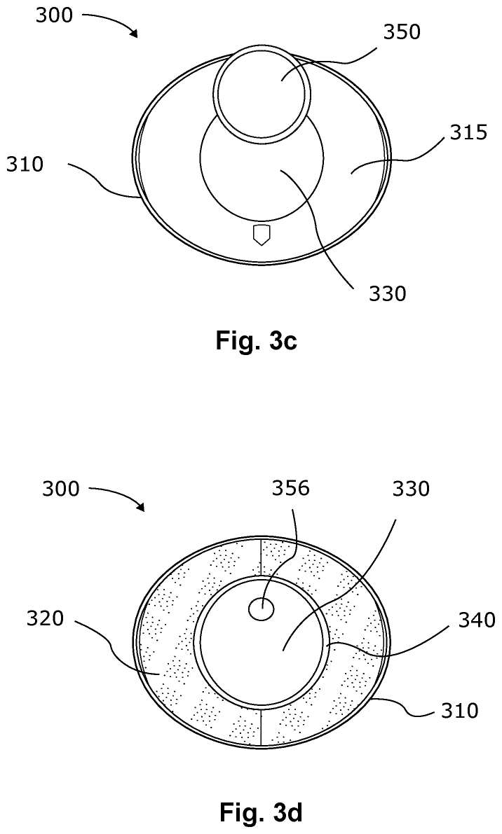

FIGS. 3a, 3b, 3c, and 3d illustrate yet another example of a non-invasive ocular delivery drug device 300. In this example, the device 300 includes a housing 310 with an active agent matrix 320 coupled thereto. Additionally, a pressure regulator 350 is coupled to a corneal dome 330 of the housing via pressure channel 356 to induce negative pressure between the housing and the eye. In this particular example, the negative pressure can be isolated to the corneal region of the device because the device includes a corneal dome 330 and a corneal seal 340 to maintain the pressure within the corneal region of the device. Additionally, in this particular example, the pressure regulator 350 can be marked, or include instructions, for applying device 300 to the eye and removing the device 300 from the eye. For example, segment 352 of the pressure regulator 350 can be marked for placement of device 300 on the eye, whereas segment 354 can be marked for removal of device 300 from the eye. In some examples, the segment 352 can forma lesser volume of the pressure regulator 350 than segment 354. As such, depressing segment 352 prior to application of the device 300 to the eye can generate sufficient negative pressure between the eye and the device 300 to couple the device 300 to the eye when segment 352 is released. Conversely, segment 354 can form a greater volume of the pressure regulator 350 than segment 352. As such, when it is desirable to remove the device 300 from the eye, depression of segment 354 can induce sufficient positive pressure between the device 300 and the eye to facilitate removal of the device 300 from the eye.

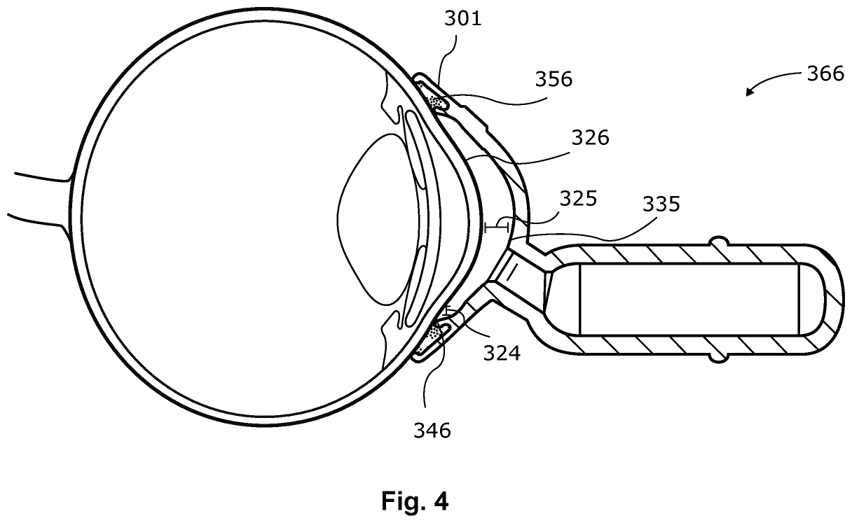

FIG. 4 illustrates an example of the device 300 coupled to an eye. As can be seen in this particular figure, a gap 325 can be maintained between an inner surface of the housing and the cornea 326 so as to minimize contact of the housing 310 with the cornea 326. Additionally, a distance 324 can be maintained between the perimeter of the cornea and the corneal seal 346 so as to maintain a fluidic seal about the cornea and minimize fluid transport across the surface of the eye to the cornea 326.

### EXAMPLES

### Example 1—Solubility Studies with Alpha-Lipoic Acid

Alpha-lipoic acid (ALA) was obtained from Sigma (Cat #T1395). The solubility of ALA was evaluated according to Table 1 below:

TABLE 1

| Material | Amount | pH | Observation |
|---|---|---|---|
| ALA (mg) | 22.1 | — | — |
| Add DI Water (ml) | 2 | 3.6 | Not dissolved |
| Add 1M NaOH (μl) | 100 | 11.7 | All dissolved |
| Add ALA (mg) | 5.7 | 6.0 | Some dissolved |
| Add 1M NaOH (μl) | 10 | 6.3 | More dissolved |
| Add 1M NaOH (μl) | 10 | 11.5 | All dissolved |
| Add ALA (mg) | 6.7 | 6.0 | Some dissolved |
| Add 1M NaOH (μl) | 15 | 9.3 | All dissolved |

In this study, the total amount of ALA added was 34.5 mg and the total volume of the solution was 2.135 ml. Thus, the solubility of ALA in water in this study was greater than 16 mg/ml. However, it was quite challenging to adjust or maintain the pH to around pH 7.4. This study shows that ALA can be dissolved in water at a concentration of greater than 16 mg/ml at a high pH and that ALA is poorly dissolved in water at a low pH at ambient temperature.

An additional solubility study was performed according to Table 2 below:

TABLE 2

| Material | Amount | pH | Observation |
|---|---|---|---|
| 0.01M PBS (m1) | 2 | 7.2 | — |
| Add ALA (mg) | 20.8 | 4.2 | Not dissolved |
| Add 1M NaOH (μl) | 80 | 7.8 | All dissolved |
| Add ALA (mg) | 10.5 | 6.3 | Mostly dissolved |
| Add 1M NaOH (μl) | 40 | 7.5 | All dissolved |
| Add ALA (mg) | 5.4 | 6.7 | Mostly dissolved |
| Add 1M NaOH (μl) | 20 | 7.9 | All dissolved |
| Add ALA (mg) | 5.1 | 6.8 | Mostly dissolved |
| Add 1M NaOH (μl) | 20 | 7.2 | All dissolved |
| Add ALA (mg) | 5.6 | 6.6 | Mostly dissolved |
| Add 1M NaOH (μl) | 20 | 7.1 | All dissolved |
| Add ALA (mg) | 10.9 | 6.2 | Mostly dissolved |
| Add 1M NaOH (μl) | 40 | 6.9 | All dissolved |
| Add ALA (mg) | 10.4 | 6.3 | Mostly dissolved |
| Add 1M NaOH (μl) | 40 | 7.1 | All dissolved |
| Add ALA (mg) | 10.5 | 6.3 | Mostly dissolved |
| Add 1M NaOH (μl) | 40 | 7.1 | All dissolved |
| Add ALA (mg) | 20.5 | 6.3 | Some dissolved |
| Add 1M NaOH (μl) | 90 | 8.3 | All dissolved |
| Add ALA (mg) | 21.5 | 6.6 | Mostly dissolved |
| Add 1M NaOH (μl) | 60 | 7.3 | All dissolved |
| Add ALA (mg) | 12.0 | 6.7 | Mostly dissolved |
| Add 1M NaOH (μl) | 40 | 7.3 | All dissolved |
| Add ALA (mg) | 21.6 | 6.7 | Mostly dissolved |
| Add 1M NaOH (μl) | 80 | 7.3 | All dissolved |
| Add ALA (mg) | 21.2 | 6.7 | Mostly dissolved |
| Add 1M NaOH (μl) | 60 | 7.1 | All dissolved |
| Add ALA (mg) | 20.5 | — | Mostly dissolved |
| Add 1M NaOH (μl) | 80 | 7.4 | All dissolved |
| Add ALA (mg) | 21.4 | — | Mostly dissolved |
| Add 1M NaOH (μl) | 60 | 7.2 | All dissolved |
| Add ALA (mg) | 23.7 | — | Mostly dissolved |
| Add 1M NaOH (μl) | 80 | 7.2 | All dissolved |
| Add ALA (mg) | 31.6 | — | Mostly dissolved |
| Add 1M NaOH (μl) | 120 | 7.3 | All dissolved |
| Add ALA (mg) | 32.1 | — | Mostly dissolved |
| Add 1M NaOH (μl) | 120 | 7.2 | All dissolved |
| Add ALA (mg) | 41.2 | — | Mostly dissolved |
| Add 1M NaOH (μl) | 160 | 7.2 | All dissolved |
| Add ALA (mg) | 42.6 | — | Mostly dissolved |
| Add 1M NaOH (μl) | 170 | 7.3 | All dissolved |

In this example, a total of 389.1 mg of ALA was dissolved in a total volume of 3.42 ml. Thus, a solubility of greater than 114 mg/ml was achieved in water at ambient temperature. With the addition of the PBS, it was easier to adjust the pH. Generally, a ratio of ALA powder in mg to 1M NaOH in μl of about 1:4 brought the pH to within about 7.0-7.4 and maintained good solubility of the ALA.

Additional studies were performed to confirm the results demonstrated in Table 2. A solubility of greater than 112 mg/ml was achieved at ambient temperature and a pH of 7.4 and a solubility of greater than 103 mg/ml was achieved at ambient temperature and a pH of 12.3. However, ALA was poorly dissolved at pH of 5.8 and below.

### Example 2—Solubility Studies with Dihydrolipoic Acid

Dihydrolipoic acid (DHLA) was obtained from Sigma (Cat #T8260). The solubility of DHLA was evaluated as presented in Table 3 below:

| Material | Amount | pH | Observation |
|---|---|---|---|
| 0.01M PBS (m1) | 1.2 | — | — |
| Add 1M NaOH (μl) | 800 | — | — |
| Add DHLA (mg) | 64.4 | — | All dissolved |
| Add DHLA (mg) | 67.0 | — | All dissolved |
| Add DHLA (mg) | 20.4 | — | All dissolved |
| Add DHLA (mg) | 58.9 | 6.5 | Mostly dissolved |
| Add 1M NaOH (μl) | 100 | 6.8 | Some dissolved |
| Add 1M NaOH (μl) | 100 | 8.5 | Mostly dissolved |

As can be seen from the results of Table 3, DHLA has a lower solubility in water than ALA. Nonetheless, a solubility of greater than 76 mg/ml and lower than 105 mg/ml was still achieved at ambient temperature. Again, the PBS helped maintain the pH within a suitable pH range.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A lipoic acid formulation, comprising:
water;
a lipoic acid (LA) agent dissolved in the water in an amount greater than or equal to about 35 mg/ml at a temperature from about 20° C. to about 28° C.; and
an amount of a buffering agent sufficient to dissolve the amount of LA agent in the water,
wherein the lipoic acid formulation has a pH of from about 6 to about 8.

2. The lipoic acid formulation of claim 1, wherein the LA agent is alpha-lipoic acid, dihydrolipoic acid, a salt thereof, or a combination thereof.

3. The lipoic acid formulation of claim 1, wherein the LA agent is dissolved in the aqueous solvent in an amount greater than or equal to 40 mg/ml.

4. The lipoic acid formulation of claim 1, wherein the LA agent is dissolved in the aqueous solvent in an amount greater than or equal to 50 mg/ml.

5. The lipoic acid formulation of claim 1, wherein the LA agent is dissolved in the aqueous solvent in an amount greater than or equal to 65 mg/ml.

6. The lipoic acid formulation of claim 1, wherein the LA agent is dissolved in the aqueous solvent in an amount greater than or equal to 75 mg/ml.

7. The lipoic acid formulation of claim 1, wherein the LA agent is dissolved in the aqueous solvent in an amount greater than or equal to 90 mg/ml.

8. The lipoic acid formulation of claim 1, wherein the LA agent is dissolved in the aqueous solvent in an amount greater than or equal to 100 mg/ml.

9. The lipoic acid formulation of claim 1, wherein the LA agent is dissolved in the aqueous solvent in an amount greater than or equal to 110 mg/ml.

10. The lipoic acid formulation of claim 1, wherein the buffering agent is present in the formulation in an amount from about 0.001 molar (M) to about 0.15 M.

11. The lipoic acid formulation of claim 1, wherein the buffering agent is a phosphate buffering agent, a borate buffering agent, a citrate buffering agent, a tromethamine buffering agent, a histidine buffering agent, or a combination thereof.

12. The lipoic acid formulation of claim 1, wherein the pH of the formulation is from about 6.5 to about 7.8.

13. An ophthalmic composition, comprising:
an amount of a lipoic acid (LA) agent sufficient to reduce disulfide bonds in a lens of an eye when administered topically to the eye;
a pharmaceutically acceptable aqueous carrier; and
an amount of a buffering agent sufficient to solubilize the amount of the LA agent in the pharmaceutically acceptable aqueous carrier,
wherein the ophthalmic composition has a pH of from about 6 to about 8 and a viscosity of less than or equal to 50 centipoise.

14. The ophthalmic composition of claim 13, wherein the amount of LA agent is from about 5 mg/ml to about 150 mg/ml.

15. The ophthalmic composition of claim 13, wherein the LA agent is alpha-lipoic acid, dihydrolipoic acid, a salt thereof, or a combination thereof.

16. The ophthalmic composition of claim 13, wherein the buffering agent is present in the composition in an amount from about 0.001 molar (M) to about 0.15 M.

17. The ophthalmic composition of claim 13, wherein the buffering agent is a phosphate buffering agent, a borate buffering agent, a citrate buffering agent, a tromethamine buffering agent, a histidine buffering agent, or a combination thereof.

18. The ophthalmic composition of claim 13, wherein the ophthalmic composition has a pH of from about 6.5 to about 7.8.

19. The ophthalmic composition of claim 13, further comprising a tonicity agent.

20. The ophthalmic composition of claim 19, wherein the tonicity agent comprises sodium chloride, potassium chloride, magnesium chloride, calcium chloride, sodium citrate, mannitol, sorbitol, dextrose, glycerin, propylene glycol, ethanol, trehalose, or a combination thereof.

21. The ophthalmic composition of claim 19, wherein the ophthalmic composition has an osmolality of from about 250 mOsm/kg to about 500 mOsm/kg.

22. The ophthalmic composition of claim 13, further comprising a preservative.

23. The ophthalmic composition of claim 22, wherein the preservative comprises a benzalkonium halides, polyquaternium-1, chlorine dioxide, benzethonium chloride, chlorobutanol, phenylmercuric acetate, phenylmercuric nitrate, thimerosal, or a combination thereof.

24. The ophthalmic composition of claim 13, wherein the ophthalmic composition is substantially free of a preservative.

25. The ophthalmic composition of claim 13, wherein the ophthalmic composition is substantially free of a thickening agent.

26. The ophthalmic composition of claim 13, wherein the ophthalmic composition is substantially free of a cyclodextrin.

27. The ophthalmic composition of claim 13, wherein the ophthalmic composition includes less than or equal to 50 particles per ml of particles having a particle size greater than or equal to 10 μm.

28. The ophthalmic composition of claim 13, wherein the ophthalmic composition includes less than or equal to 5 particles per ml of particles having a particle size greater than or equal to 25 μm.

29. The ophthalmic composition of claim 13, wherein the ophthalmic composition includes less than or equal to 2 particles per ml of particles having a particle size greater than or equal to 50 μm.
